(19) United States
(12) Patent Application Publication
(10) Pub. No.: US 2010/0272811 A1
(43) Pub. Date: Oct. 28, 2010

(54) COMPLEX OF TROSPIUM AND PHARMACEUTICAL COMPOSITIONS THEREOF

(75) Inventors: David S. Scher, Hudson, MA (US); Rachel A. Ryznal, North Oxford, MA (US); Charles D. Blizzard, Westwood, MA (US)

Correspondence Address: ELMORE PATENT LAW GROUP, PC, 515 Groton Road, Unit 1R, Westford, MA, 01886, US

(73) Assignee: Alkermes,Inc., Cambridge, MA (US)

(21) Appl. No.: 12/507,943

(22) Filed: Jul. 23, 2009

Related U.S. Application Data
(60) Provisional application No. 61/083,104, filed on Jul. 23, 2008.

(51) Int. Cl. A61K 9/14; A61K 31/46; A61P 13/10; A61P 11/00

(52) U.S. Cl. 424/489; 514/278

(57) ABSTRACT

The invention is directed to a complex of trospium and saccharin. In one embodiment, the complex is a crystalline form. In another embodiment, the complex is a monohydrate form. The invention also encompasses methods of preparing the the saccharin complex of trospium and to pharmaceutical compositions thereof.

### RELATED APPLICATION

This application claims the benefit of U.S. Provisional Application No. 61/083,104, filed on Jul. 23, 2008. The entire teachings of the above application are incorporated herein by reference.

### BACKGROUND OF THE INVENTION

Trospium (endo-3-[(hydroxydiphenyl-acetyl)oxy]spiro[8-azoniabicyclo[3.2.1]octane-8,-1′-pyrrolidinium]chloride) is an anticholinergic agent that acts as an antagonist of muscarinic acetylcholine receptors. Trospium chloride is sold under the trade name SANCTURA® and is approved as an oral dosage form for the treatment of overactive bladder. Trospium chloride has also been described as useful for the treatment of pulmonary conditions such as interstitial cystitis, asthma, acute respiratory distress syndrome (ARDS), cystic fibrosis as well as chronic obstructive pulmonary disease (COPD).

Trospium chloride has a relatively high aqueous solubility. It would be advantageous to prepare a formulation of trospium that has a lower aqueous solubility than trospium chloride. Trospium formulations with lower aqueous solubility may display a modified pharmacokinetic profile and/or have improved stability and/or be associated with reduced bitter taste when administered orally when compared to trospium chloride.

### SUMMARY OF THE INVENTION

The present invention is directed to a complex of trospium and saccharin and pharmaceutical compositions thereof. In certain embodiments, the complex is a crystalline form. In another embodiment, the complex is a monohydrate. In other embodiments, the complex is a particulate.

The invention is additionally directed to a method of preparing the saccharin complex of trospium.

In a further embodiment, the invention is directed to pharmaceutical compositions comprising trospium chloride and saccharin or a salt thereof.

In yet another embodiment, the invention is directed to a method of treating a patient having a condition that is alleviated or ameliorated by inhibiting a muscarinic acetylcholine receptor comprising administering a complex of trospium and saccharin or a pharmaceutical composition thereof.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A is an overlay of XRPD patterns obtained for trospium containing formulated precipitate and powders: trospium saccharin precipitate; 30% (w/w) trospium/15% (w/w) sodium saccharin/55% (w/w) leucine; 10% (w/w) trospium/5% (w/w) sodium saccharin/85% (w/w) leucine; 10% (w/w) trospium/5% (w/w) sodium saccharin/10% (w/w) sodium citrate/75% (w/w) leucine and 10% (w/w) trospium/5% (w/w) acid saccharin/85% (w/w) leucine formulated powders.

FIG. 1B is an overlay of XRPD patterns obtained for saccharin, trospium saccharin, sodium saccharin and trospium chloride.

FIG. 1C is the XRPD pattern for trospium saccharin precipitate.

FIG. 2 is a drawing of the crystal structure for the trospium saccharin precipitate.

FIG. 3A shows thermogravimetric (TGA) and Differential Scanning Thermogram (DSC) overlays for the trospium saccharin precipitate.

FIG. 3B is a thermal analysis profile obtained for trospium saccharin precipitate and formulations comprising trospium and saccharin (30%, 10% and 4% based on weight percent of trospium chloride in the formulation).

FIGS. 4A, 4B and 4C are plots showing percent change in emitted size (VMGD) post RH equilibration at 20%, 40% and 60% RH at 25° C. followed by storage at 50° C. and 60° C., respectively. The “low ethanol,” “saccharin,” “acetone,” and “citrate” formulation are described in detail in Example 4.

FIG. 5 is a plot of plasma concentration (ng/ml) of trospium over time (minutes) in rat administered trospium or trospium saccharin complex via insufflation.

FIG. 6 is a plot of mean FEV₁ change from baseline versus time of groups treated with 100 μg TrIP-2D formulation (2% TrCl (100 μg) formulated with leucine and 1,2-dipalmitoyl-sn-glycero-3-phosphocholine (DPPC)), 400 ug TrIP-2D, 100 μg TrIP-2SS (2% TrCl (100 μg) formulated with leucine and sodium saccharin) and 100 μg TrIP-2SS and 12 μg Foradil. Each data point represents the mean of 24 subjects.

### DETAILED DESCRIPTION OF THE INVENTION

As used herein, the word “a” or “an” is meant to encompass one or more unless otherwise specified.

Percentages or “%” in reference to components of a pharmaceutical composition or formulation are percentages by weight of the composition unless otherwise indicated.

The invention is directed to a novel complex of trospium and saccharin. In certain embodiments, the complex is a salt. In additional embodiments, the complex is a crystalline form.

The invention is additionally directed to pharmaceutical compositions comprising trospium chloride and saccharin. In one embodiment, the pharmaceutical composition comprises a complex of trospium and saccharin and a pharmaceutically acceptable carrier or excipient. In other embodiments, the pharmaceutical composition is a dry powder.

The invention also encompasses methods for the preparation of a complex of saccharin and trospium.

In other embodiments, the invention relates to a complex formed by combining trospium chloride and saccharin or a salt thereof. Exemplary salts of saccharin include sodium, potassium, calcium and ammonium salts.

The terms “complex” or “complexes”, as used herein, unless otherwise indicated, refer to an acid-base pair that has a defined stoichiometry and contains ionized, un-ionized and/or partially charged base and acid species, wherein the extent of proton transfer from acid (proton donor) to the base (proton acceptor) can vary in proportions from none, to partial, to all. One of ordinary skill in the art will appreciate that the above definition of “complex” includes salts. The term “trospium saccharin complex” refers to a complex of trospium and saccharin. In one embodiment of the invention, the complex is a saccharin salt of trospium. As used herein, the term “trospium saccharin salt” refers to the saccharin salt of trospium.

As used herein, “crystalline” or “crystal” refers to a solid having a highly regular chemical structure. Crystalline trospium saccharin complex can be a single crystalline form of trospium saccharinate, or a mixture of different single crystalline forms. A single crystal form means a single crystal or a plurality of crystals in which each crystal has the same crystal form.

The trospium saccharin complex can be an amorphous form, a crystalline form or a partially crystalline form. The invention also encompasses anhydrates, hydrates and solvates of trospium saccharin complex. In one embodiment, the trospium saccharin complex is a crystalline form. In another embodiment, the trospium saccharin complex is a monohydrate. In an additional embodiment, the trospium saccharin complex is a particulate. In yet another embodiment, the trospium saccharin complex is a dry powder.

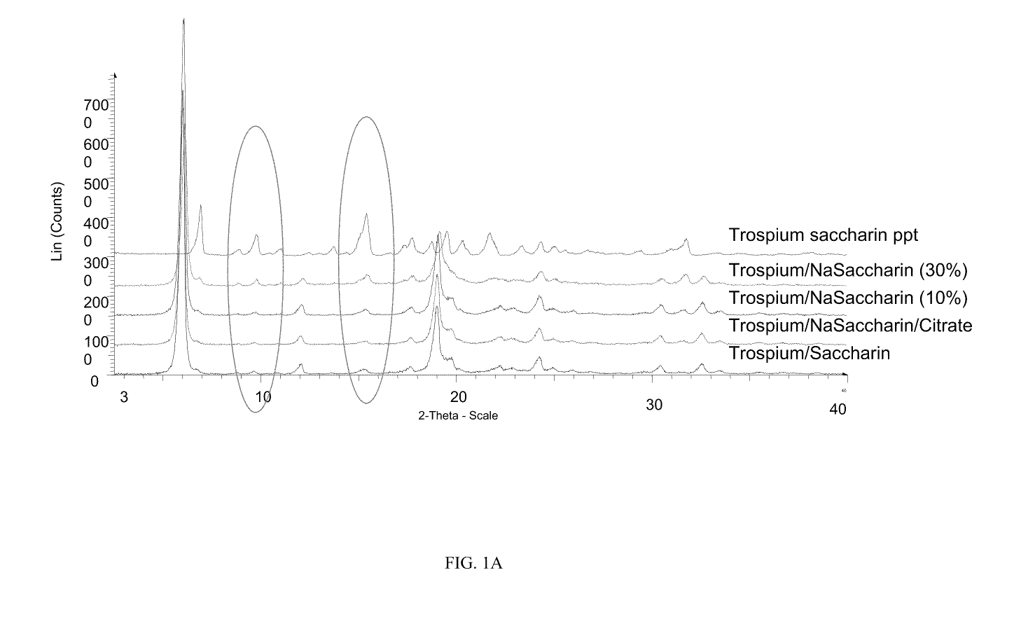

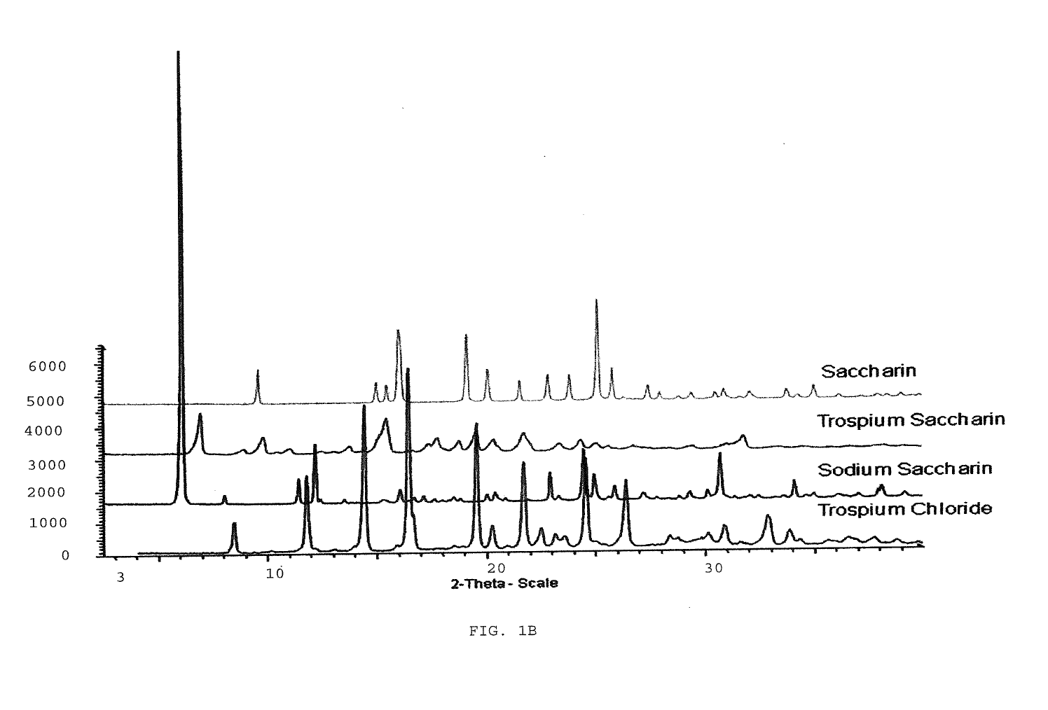

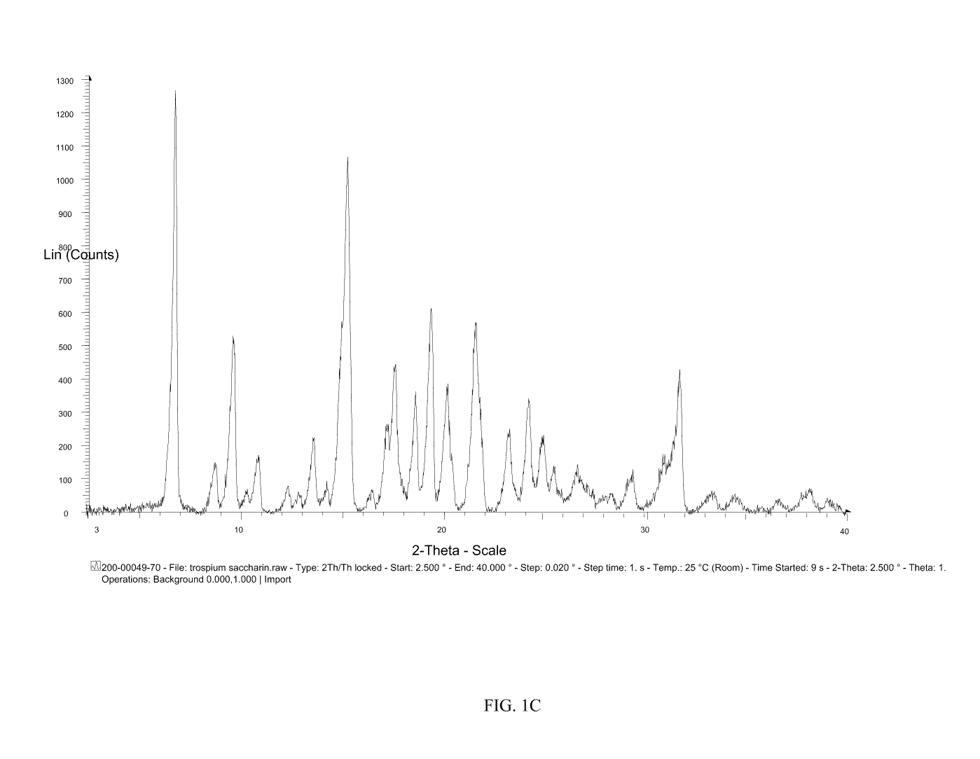

In certain aspects of the invention, the trospium saccharin complex is characterized by the X-ray powder diffraction (XRPD) pattern shown in FIG. 1C with values of 2θ angles and relative intensities shown there. In one embodiment, the trospium saccharin complex has at least one major XRPD peak selected from 9.7, 15.4, 19.5 and 21.6 degrees 2θ±0.2 degrees 2θ. In another embodiment, the trospium saccharin complex is characterized by at least two major XRPD peaks selected from 9.7, 15.4, 19.5 and 21.6 degrees 2θ±0.2 degrees 2θ. In an additional embodiment, the trospium saccharin complex is characterized by at least four major peaks at 9.7, 15.4, 19.5 and 21.6 degrees 2θ±0.2 degrees 2θ. In a further embodiment, the trospium saccharin complex is characterized by two major XRPD peaks at 9.7 and 15.4 degrees 2θ±0.2 degrees 2θ. In yet another embodiment, the trospium saccharin complex has at least two of the following XRPD peaks: 6.8, 8.9, 9.7, 11.0, 13.7, 15.4, 17.4, 17.8, 18.7, 19.5, 20.2, 21.6, 23.5, 24.3, 25.2 and 31.5±0.2 degree 2θ. In a further embodiment, the trospium saccharin complex is characterized by the following XRPD peaks: 6.8, 8.9, 9.7, 11.0, 13.7, 15.4, 17.4, 17.8, 18.7, 19.5, 20.2, 21.6, 23.5, 24.3, 25.2 and 31.5±0.2 degree 2θ. As used herein, “major XRPD peak” refers to an XRPD peak with a relative intensity greater than 25%. Relative intensity is calculated as a ratio of the peak intensity of the peak of interest versus the peak intensity of the largest peak.

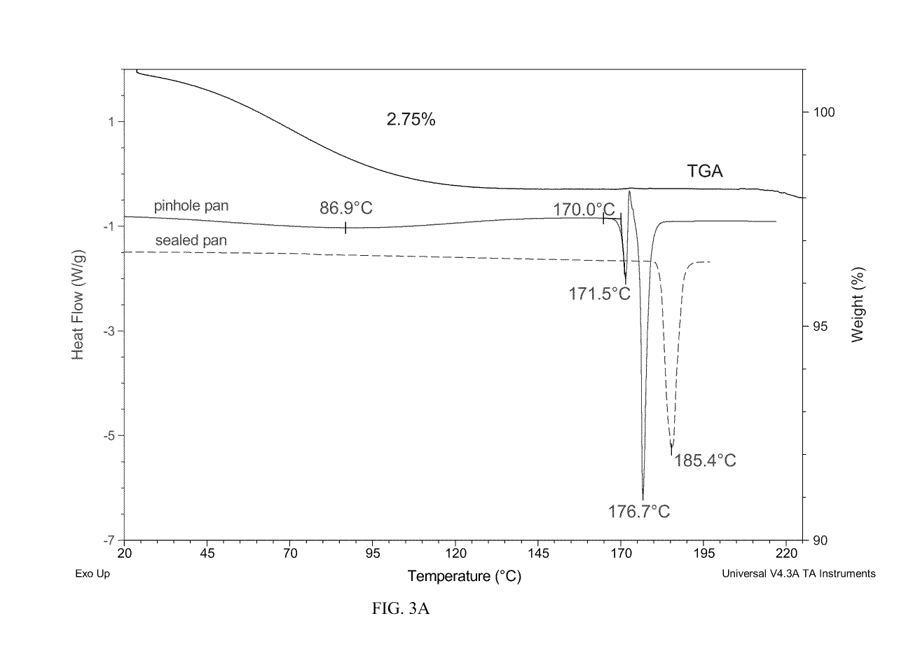

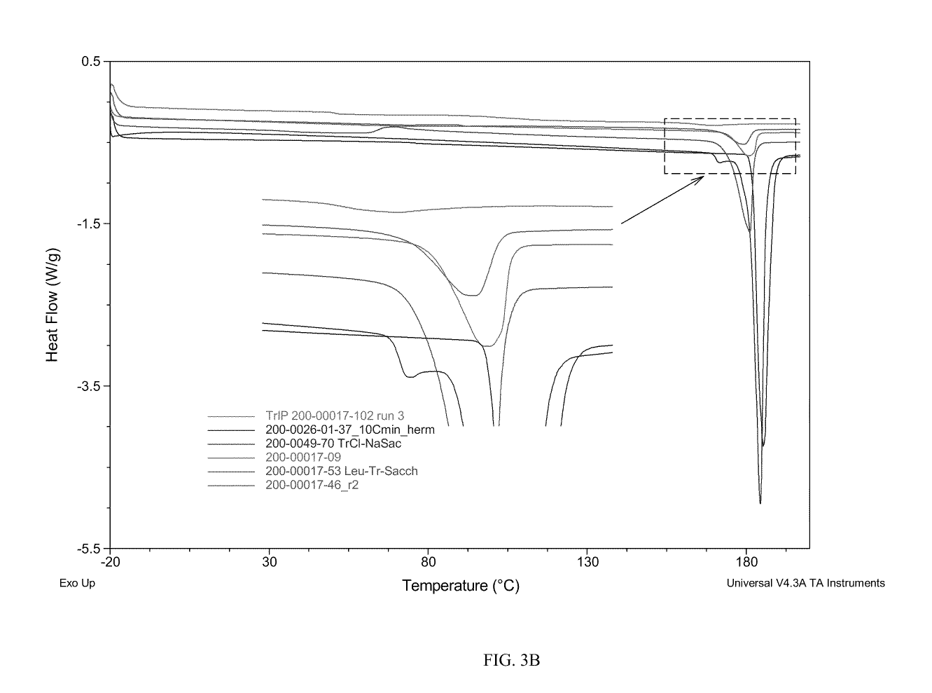

In an additional embodiment, the trospium saccharin complex is characterized by a melting onset at about 170° C. and two endothermic peaks at about 176° C. and 185° C. at differential scanning calorimetry (“DSC”) profile using a sample pan configuration which allows the water to evaporate. When a hermetic sample pan configuration is used, the trospium saccharin complex is characterized by a single endothermic transition at about 185° C. The DSC and TGA for the trospium saccharin complex is shown in FIGS. 3A and 3B which shows heat flow and weight change as a function of temperature from trospium saccharin complex. The DSC is performed on the sample using a scanning rate of 10° C./minute. In another embodiment, the trospium saccharin complex in the formulation is characterized by a single endothermic transition between about 160° C. and 185° C. in the DSC profile. In another embodiment, the trospium saccharin complex is characterized by a single endothermic transition at 185±0.5° C. in the DSC profile.

In additional embodiments, the trospium saccharin complex is characterized by a combination of one or more of the XRPD and DSC equilibration measurements described above.

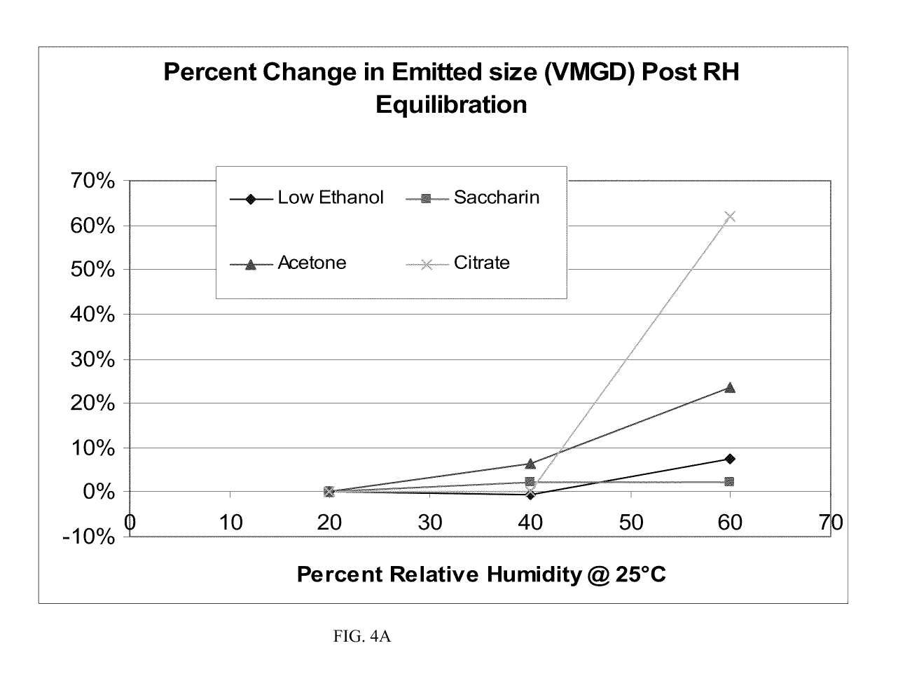

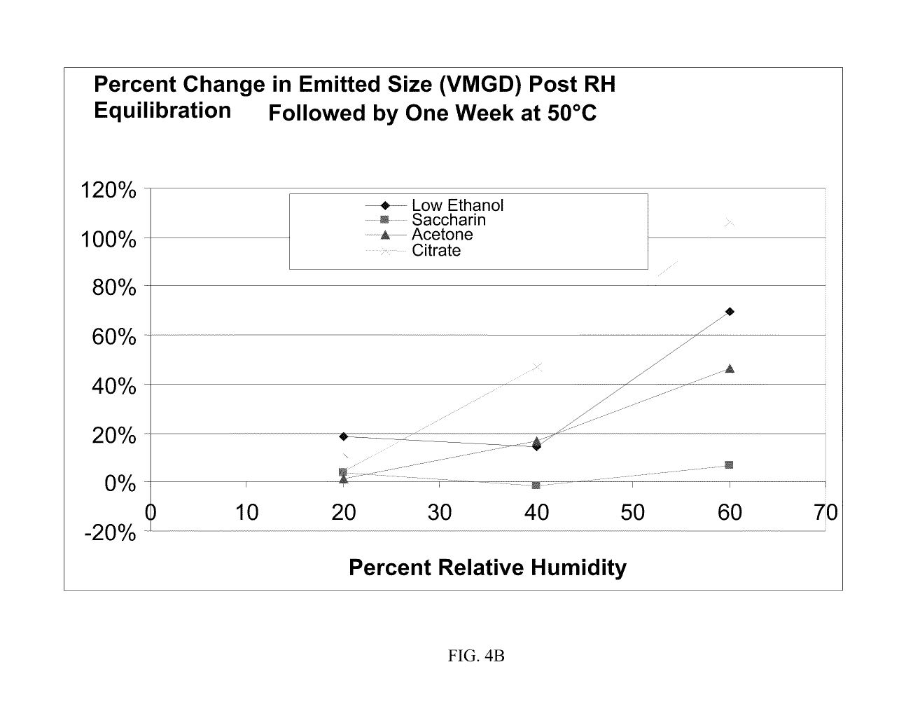

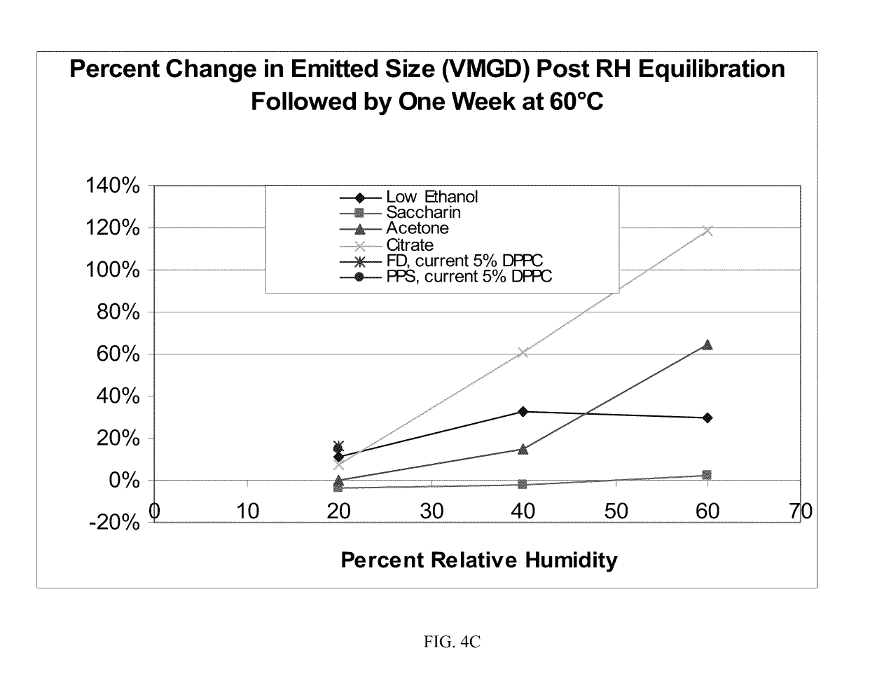

In a further embodiment, the invention is a formulation comprising the trospium saccharin complex wherein the formulation is characterized by relative humidity (“RH”) equilibration profiles shown in FIGS. 4A, 4B and 4C. The profiles show the change in particle size of a sample of trospium chloride and sodium saccharin as the relative humidity of the environment changes from 20% to 40% and 60% at the temperatures of 25° C., 50° C. and 60° C. After RH challenge, the profile for the formulation the trospium saccharin complex shows no significant percent change in the diameter of the spray-dried particles (VMGD) at 20, 40 or 60% at 25° C. RH equilibration.

In an additional embodiment, the invention is directed to a pharmaceutical composition comprising saccharin or a salt thereof and a therapeutically effective amount of trospium chloride and a pharmaceutically acceptable carrier or excipient. In other embodiments, the invention is directed to pharmaceutical compositions comprising a therapeutically effective amount of a complex of trospium and saccharin and a pharmaceutically acceptable carrier or excipient. In one embodiment, the invention is a pharmaceutical composition comprising a therapeutically effective amount of trospium saccharin salt and a pharmaceutically acceptable carrier or excipient.

A “therapeutically effective amount” is an amount which, alone or in combination with one or more other active agents, can control, decrease, inhibit, ameliorate, prevent or otherwise affect one or more symptoms of a disease or condition to be treated. In certain embodiments, the composition comprises from about 0.5 to about 30% by weight trospium saccharin complex. In another embodiment, the composition comprises from about 0.5 to about 10% trospium saccharin complex. In an additional embodiment, the composition comprises trospium saccharin complex in an amount from about 0.5 to about 5%. In one embodiment, the composition comprises trospium saccharin complex in an amount of about 1%. In yet another embodiment, the composition comprises trospium saccharin complex in an amount of about 5%. In a further embodiment, the composition comprises trospium saccharin complex in an amount of about 10%.

In other aspects of the invention, the pharmaceutical composition of the invention is a powder or particulate. In an additional embodiment, the pharmaceutical composition is a dry powder. The dry powder can be adapted for administration with a dry powder inhaler. As used herein, a “dry powder” contains less than about 5% by weight of water, based on the total weight of the solids in the composition. In yet another embodiment, the composition comprises micronized trospium saccharin complex. In an additional embodiment, the composition comprises spray-dried trospium saccharin complex.

In other aspects, the powder possesses aerosol characteristics that permit effective delivery of the particles to the respiratory system. As will be understood by one of skill in the art, aerosol performance can be evaluated based on parameters including geometric diameter, aerodynamic diameter, density and fine particle fraction. These characteristics have been described, for example, in U.S. Patent Publication No. 2004/0042970, the contents of which are incorporated by reference herein.

In certain embodiments, the powder that has a density of less than about 0.4 g/cm³, or less than about 0.3 g/cm³, or less than about 0.2 g/cm³, or less than about 0.1 g/cm³ or between about 0.05 g/cm³ and about 0.4 g/cm³.

In other embodiments, the powder has a mass mean aerodynamic diameter (MMAD) of less than about 5.8 microns. In another embodiment, the particles have a MMAD from about 1 to about 5.8 microns. In another embodiment, the particles have a MMAD from about 1 to about 3 microns. In another aspect, the particles have a MMAD from about 2 to about 4 microns. In yet another embodiment, the particles have a MMAD from about 3 to about 5.8 microns.

Fine particle fraction can be used as another way to characterize the aerosol performance of a dispersed powder. Fine particle fraction describes the size distribution of airborne particles. Gravimetric analysis, using cascade impactors, is one method of measuring the fine particle fraction of airborne particles. A two-stage collapsed ACI can be used to measure fine particle fraction. The two-stage collapsed ACI consists of only the top two stages of the eight-stage ACI and allows for the collection of two separate powder fractions. The ACI is made up of multiple stages consisting of a series of nozzles and an impaction surface. At each stage, an aerosol stream passes through the nozzles and impinges upon the surface. Particles in the aerosol stream with a large enough inertia will impact upon the plate. Smaller particles that do not have enough inertia to impact on the plate will remain in the aerosol stream and be carried to the next stage. In one embodiment, the particles of the invention are characterized by fine particle fraction. A two-stage collapsed Andersen Cascade Impactor is used to determine fine particle fraction. Specifically, a two-stage collapsed ACI is calibrated so that the fraction of powder that is collected on stage one is composed of particles that have an aerodynamic diameter of less than 5.8 microns and greater than 3.3 microns. The fraction of powder passing stage one and depositing on a collection filter is thus composed of particles having an aerodynamic diameter of less than 3.3 microns. The airflow at such a calibration is approximately 60 L/min. The terms “FPF(<5.8)” and “fine particle fraction, less than 5.8 microns,” as used herein, refer to the fraction of a sample of particles that have an aerodynamic diameter of less than 5.8 microns. FPF(<5.8) can be determined by dividing the mass of particles deposited on the stage one and on the collection filter of a two-stage collapsed ACI by the mass of particles weighed into a capsule for delivery to the instrument. The terms “FPF (<3.3)” and “fine particle fraction, less than 3.3 microns,” as used herein, refer to the fraction of a mass of particles that have an aerodynamic diameter of less than 3.3 microns. FPF(<3.3) can be determined by dividing the mass of particles deposited on the collection filter of a two-stage collapsed ACI by the mass of particles weighed into a capsule for delivery to the instrument. The FPF(<5.8) has been demonstrated to correlate to the fraction of the powder that is able to make it into the lungs of the patient, while the FPF(<3.3) has been demonstrated to correlate to the fraction of the powder that reaches the deep lung of a patient. These correlations provide a quantitative indicator that can be used for particle optimization.

In one embodiment, a mass of particles of the invention has a FPF(<5.8) of at least about 40%. In another embodiment, a mass of particles of the invention has a FPF(<5.8) of greater than about 50%. In yet another embodiment, a mass of particles has a FPF(<5.8) of greater than about 60%. In an additional embodiment, a mass of particles have a FPF (<3.3) of greater than about 10%. In another embodiment, a mass of particles have a FPF (<3.3) greater than about 20%. In a further embodiment, a mass of particles have a FPF (<3.3) greater than about 50%.

The pharmaceutical composition additionally comprises one or more pharmaceutically acceptable carriers or excipients. As used herein, the term “pharmaceutically acceptable carrier or excipient” means any non-toxic diluent or other formulation auxiliary that is suitable for use in a combination of the invention. Examples of pharmaceutically acceptable carriers or excipients include but are not limited to solvents, cosolvents, solubilizing agents (such as sorbitol, glycerin or cyclodextrin), bulking agents, amino acids, sugars, polysaccharides, salts, buffers, lipids, cholesterol, fatty acid, tablet binders, fillers, preservatives, tablet disintegrating agents, flow regulating agents, plasticizers, wetting agents, dispersing agents, emulsifiers, pH altering additives, flavor masking agents, flavorings, sweeteners and combinations thereof.

In some embodiments, the composition comprises a flavor masking agent and/or a flavoring agent and/or a sweetener. Exemplary flavor masking agents, flavoring agents and sweeteners that can be used in the pharmaceutical composition include citric acid, sodium citrate, and sugars such as polyalditol, aspartame and sucralose.

In certain embodiments, the pharmaceutical composition additionally comprises agents that provide improvements in powder handling, such as sodium citrate.

The pharmaceutical composition can also comprise one or more bulking agents. Examples of bulking agents are well-known in the art and include amino acids, non-reducing sugars, polyhydric alcohols, dipeptides and tripeptides. Exemplary non-reducing sugars include trehalose, sucrose and lactose. Exemplary polyhydric alcohols include sorbitol, xylitol, mannitol and polyalditol.

In one embodiment, the bulking agent is an amino acid. In further embodiments, the amino acid is a hydrophobic amino acid. Hydrophobic amino acids include, for example, leucine, isoleucine, cysteine, alanine, methionine, phenylalanine, proline, tryptophan, tyrosine and valine. In another embodiment, the amino acid is leucine. The amino acid can be included in the composition in an amount between about 10% to about 99.5% by weight of total composition. In yet another embodiment, the amino acid is included in the composition in an amount of at least about 50% by weight of the composition. In an additional embodiment, the amino acid is included in an amount of at least about 70% by weight of the composition. In another embodiment, the amino acid is included in an amount of at least about 85% by weight of the composition. In a further embodiment, the amino acid is included in the composition in an amount between about 85 and about 99% by weight. As used herein, the term “by weight of the composition” or “by weight” does not include the weight of water and/or residual solvents and/or volatiles.

The particles and respirable compositions comprising the particles of the invention can additionally comprise a phospholipid or a combination of phospholipids.

Examples of suitable phospholipids include, among others, those listed and described in U.S. Patent Publication No. 2001/0036481A1. The contents of this application are incorporated by reference in their entirety. Other suitable phospholipids include phosphatidylcholines, phosphatidylethanolamines, phosphatidylglycerols, phosphatidylserines, phosphatidylinositols and combinations thereof. Specific examples of phospholipids include but are not limited to 1,2-dipalmitoyl-sn-glycero-3-phosphocholine (DPPC), 1,2-distearoyl-sn-glycero-3-phosphocholine (DSPC), 1-myristoyl,-2-stearoyl-sn-glycero-3-phosphocholine (MSPC), 1,2-dimyristoyl-sn-glycero-3-phosphoethanolamine (DMPE), 1,2-distearoyl-sn-glycero-3-[phospho-rac-(1-glycerol)] (DSPG), 1,2-dipalmitoyl-sn-glycero-3-phosphoethanolamine (DPPE), or any combination thereof. Other phospholipids are known to those skilled in the art. In an additional embodiment, the phospholipids are endogenous to the lung. In another embodiment, the phospholipid is included in an amount between about 1% and about 70%. In yet another embodiment, the phospholipid is included in an amount between about 1% and about 30%. In a further embodiment, the phospholipid is included in an amount between about 5% and about 10% by weight of the total composition.

It is understood that the composition is made of or contains salts that may disassociate and/or reassociate with the original or an alternative ion during processing, manufacture or storage. Thus, where the composition is said to “comprise” one or more such salts, it is intended to include compositions possessing one or more stated salts and also the components of each salt in a disassociated and/or reassociated state. Such disassociation and/or reassociation can be partial or complete. For example, a composition comprising trospium chloride and sodium saccharin may reassociate during processing, manufacture or storage to additionally or alternatively comprise, or even consist of, trospium saccharin and sodium chloride. While it is generally understood that the composition will preferably be made from the stated materials (e.g., trospium chloride and sodium saccharin is added to the process), it is possible that the composition can also be obtained from adding alternative combinations of the ions (e.g., trospium saccharin and sodium chloride or trospium free base, saccharin free base, hydrogen chloride and sodium hydroxide). All such alternatives are intended to be embraced by this terminology as defined herein. That is, “compositions comprising” the stated salt components (e.g., trospium chloride and sodium saccharin), as defined herein, includes the composition made by adding trospium saccharin and sodium chloride.

In some embodiments, the composition comprises from about 0.5 to about 15% trospium chloride, from about 0.1 to about 10% sodium saccharin and from about 75 to about 99.4% leucine by weight of the composition. In yet another embodiment, the composition comprises from about 0.5 to about 10%, from about 0.1 to about 5% sodium saccharin and from about 85 to about 99.4% leucine. In a further embodiment, the composition comprises from about 0.5 to about 5% trospium chloride, from about 0.5 to about 2% sodium saccharin and from about 93 to about 99% leucine. In an additional embodiment, the composition comprises about 4% trospium chloride, about 2% sodium saccharin and about 94% leucine. In yet another embodiment, the composition comprises about 2% trospium chloride, about 1% sodium saccharin and about 97% leucine. In an additional embodiment, the composition comprises about 1% trospium chloride, about 0.5% sodium saccharin and about 98.5% leucine.

In additional embodiments of the invention, the composition comprises from about 5 to about 15% trospium chloride, from about 2 to about 10% sodium saccharin and from about 75% to about 93% leucine by weight of the composition. The composition can also comprise from about 7 to about 12% trospium chloride, from about 3 to about 8% sodium saccharin and about 80% to about 90% leucine by weight of the compositions. In a further embodiment, the composition comprises about 10% trospium chloride, about 5% sodium saccharin and about 85% leucine by weight of the composition.

The present invention also encompasses methods for the preparation of trospium saccharin complex. In one embodiment, the method comprises reacting trospium chloride with saccharin or a salt thereof and recovering trospium saccharin complex. In another embodiment, an aqueous solution of trospium chloride is combined with an aqueous solution of a saccharin salt and the trospium saccharin complex is recovered. The solution is optionally cooled before recovery of trospium saccharin complex. In a further embodiment, the saccharin or salt thereof and the trospium chloride are mixed at a ratio between about 2:1 to about 1:2. In another embodiment, the saccharin or salt thereof and the trospium chloride are mixed at a ratio of about 1:1.

The trospium saccharin complex is optionally purified after recovery from the reaction mixture. In one embodiment, the trospium saccharin complex is substantially pure. As used herein, substantially pure trospium saccharin complex has a purity greater than 90% by weight, including greater than about 91, 92, 93, 94, 95, 96, 97, 98 and 99%, by weight based on the weight of the complex together with reaction impurities and/or processing impurities. The presence of reaction impurities and/or processing impurities may be determined by analytical techniques known in the art, such as, for example, chromatography, nuclear magnetic resonance spectroscopy, mass spectrometry, or infrared spectroscopy.

The trospium chloride and saccharin or salt thereof can be combined to prepare a powder or particulate form of trospium saccharin complex during spray-drying. Suitable spray-drying techniques are described, for example, by K. Masters in “Spray Drying Handbook”, John Wiley & Sons, New York (1984). Generally, during spray-drying, heat from a hot gas, such as heated air or nitrogen, is used to evaporate a solvent from droplets formed by atomizing a continuous liquid feed. An organic solvent or a co-solvent comprising aqueous and organic solvents can be employed to form a feed for spray-drying the particles of the present invention. Suitable organic solvents that can be employed include but are not limited to alcohols such as, for example, ethanol, methanol, propanol, isopropanol and butanol. Other organic solvents include, but are not limited to, perfluorocarbons, dichloromethane, chloroform, ether, ethyl acetate, methyl tert-butyl ether and others. Co-solvents that can be employed include an aqueous solvent and an organic solvent. Aqueous solvents include water and buffered solutions. In one embodiment, an ethanol/water solvent is utilized. In another embodiment, the ethanol to water ratio ranges from about 90:10 to about 10:90, by volume. In yet another embodiment, the ethanol to water ratio ranges from about 70:30 to about 30:70, by volume.

The mixture (comprising the solvent/co-solvent, trospium chloride, saccharin and optionally one or more additional agents) can have a neutral, acidic or alkaline pH. Optionally, a pH buffer can be added to the solvent or co-solvent or to the formed mixture. The pH can range from about 5 to about 8. In one embodiment, organic soluble particle components are dissolved in an organic phase and water soluble particle components are dissolved in an aqueous phase. The solutions are heated as necessary to assure solubility. In another embodiment, ethanol soluble particle components are dissolved in an ethanol phase and water soluble particle components are dissolved in an aqueous phase.

In one aspect of the present invention, a hydrophilic component and a hydrophobic component are prepared. The hydrophobic and hydrophilic components are then combined in a static mixer to form a combination. The combination is atomized to produce droplets, which are dried to form dry particles. In one aspect of this method, the atomizing step is performed immediately after the components are combined in the static mixer.

In a further aspect of the present invention, a method for preparing a dry powder composition is provided. In such a method, first and second components are prepared, one or both of which comprise the trospium saccharin complex and optionally an additional active agent. The first and second components are combined in a static mixer to form a combination. In one embodiment, the first and second components are physically and/or chemically incompatible with each other. In one aspect, the first and second components are such that the combination step causes degradation in one of the components. In another aspect, a material present in the first component is incompatible with a material present in the second component. The combination is atomized to produce droplets that are dried to form dry particles. In another aspect of such a method, the first component comprises trospium chloride and optionally an additional active agent and optionally one or more excipients dissolved in an aqueous solvent, and the second component comprises saccharin and optionally further comprises an additional active agent and/or one or more excipients, dissolved in an organic solvent. In yet another aspect of the invention, the first component comprises a saccharin salt and optionally comprises an additional active agent and/or one or more excipients, dissolved in an aqueous solvent, and the second component comprises trospium chloride and optionally comprises an additional active agent and/or one or more excipients, dissolved in an organic solvent. In another aspect of the invention, the first component comprises trospium chloride and optionally comprises an additional active agent and/or a saccharin salt and/or one or more excipients, dissolved in an aqueous solvent, and the second component comprises saccharin and optionally comprises an additional active agent and/or one or more excipient dissolved in an organic solvent. One or more of the solutions can be heated to assure solubility of the components.

In one embodiment, the apparatus used for practice of the present invention includes a static mixer having an inlet end and an outlet end (e.g., a static mixer as more fully described in U.S. Pat. No. 4,511,258, the contents of which are incorporated in their entirety herein by reference, or other suitable static mixers such as, but not limited to, Model 1/4-21, made by Koflo Corporation). The static mixer is used to combine an aqueous component with an organic component to form a combination. Means are provided for transporting the aqueous component and the organic component to the inlet end of the static mixer. In one aspect, the aqueous and organic components are transported to the static mixer at substantially the same rate. An atomizer in fluid communication with the outlet end of the static mixer can be used to atomize the combination into droplets. The droplets can then be dried in a dryer to form dry particles. In a further embodiment, the apparatus used to practice the present invention includes a geometric particle sizer that determines a geometric diameter of the dry particles, and an aerodynamic particle sizer that determines an aerodynamic diameter of the dry particles.

Methods and devices suitable for forming particles of the present invention are discussed in U.S. Pat. No. 7,008,644, the contents of which are incorporated by reference herein.

Spray-drying solutions prepared as described above can be fed to a drying vessel. For example, a nozzle or a rotary atomizer can be used to distribute the solutions to the drying vessel. In one embodiment, a rotary atomizer is employed, such as a vaned rotary atomizer such as a rotary atomizer having a 4- or 24-vaned wheel. A non-limiting example of a spray-dryer that uses rotary atomization is the Mobile Minor Spray Dryer, manufactured by Niro, Inc. (Denmark). Actual spray-drying conditions will vary depending in part on the composition of the spray-drying solution and material flow rates. In some embodiments, the inlet temperature to the spray-dryer is about 100 to about 200° C. In some embodiments, the inlet temperature is about 110 to about 160° C. The spray-dryer outlet temperature will vary depending upon such factors as the feed temperature and the properties of the materials being dried. In one embodiment, the outlet temperature is about 35 to about 80° C. In another embodiment, the outlet temperature is about 45 to about 70° C., such as for example about 45 to about 65° C., or about 60 to about 70° C.

In one embodiment, trospium saccharin complex or a pharmaceutical composition thereof can be used to treat a patient having a condition that is alleviated or ameliorated by inhibiting a muscarinic acetylcholine receptor. In another embodiment, trospium saccharin complex can be used to treat a smooth muscle hyperactivity disorder. Smooth muscle hyperactivity disorders include, for example, overactive bladder and pollakiuria, and gastrointestinal hyperactivity, and other smooth muscle hyperactivity, urolithiasis, cholelithiasis, choledocholithiasis and smooth muscle hyperactivity disorder occurring in conjunction with asthma. In a further embodiment, the invention is directed to a method of treating a patient suffering from a condition selected from the group consisting of acute lung injury (ALI), acute respiratory distress syndrome (ARDS), chronic obstructive pulmonary, airways or lung disease (COPD, COAD or COLD, respectively), chronic bronchitis, emphysema, bronchiectasis and exacerbation of airway hyperreactivity consequent to other drug therapy, in particular other inhaled drug therapy, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, overactive bladder, and interstitial cystitis. In a particular embodiment, the condition is COPD.

In one embodiment, the trospium saccharin complex is administered using an inhalation device. In a further embodiment, the total daily dose of the trospium saccharin complex administered to a subject can be in amounts, for example, from 0.01 to 50 μg/kg body weight or more usually from 0.1 to 25 μg/kg body weight. In general, treatment regimens according to the present invention comprise administration to a patient in need of such treatment from about 20 μg to about 1200 μg of the trospium saccharin complex disclosed herein per day in single or multiple doses. In an additional embodiment, the amount of trospium saccharin complex administered is about 20, 40, 60, 80, 100, 120, 140, 160, 180, 200, 220, 240, 260, 280, 300, 320, 340, 360, 380, 400, 420, 440, 460, 480, 500 μg per inhalation. In a preferred embodiment, the amount of trospium saccharin complex administered is about 100 to about 400 micrograms per inhalation.

The specific dose level for any particular patient will vary depending upon a variety of factors, including but not limited to, the activity of the specific therapeutic agent employed; the age, body weight, general health, sex and diet of the patient; the time of administration; the rate of excretion; drug combination; the severity of the particular disease being treated; and the form of administration. Typically, in vitro dosage-effect results provide useful guidance on the proper doses for patient administration. Studies in animal models are also helpful. The considerations for determining the proper dose levels are well known in the art.

Dosing schedules may be adjusted to provide the optimal therapeutic response. For example, administration can be one to three times daily for a time course of one day to several days, weeks, months, and even years, and may even be for the life of the patient. Practically speaking, a unit dose of the trospium saccharin complex can be administered in a variety of dosing schedules, depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, every other day, three times weekly, twice weekly, once weekly, twice monthly, once monthly, and so forth. Dosing may be provided alone or in combination with other drugs and may continue as long as required for effective treatment of the disease or disorder as described herein.

The composition may additionally comprise one or more additional active agents. As used herein, an active agent is defined as a small molecule or biologic with pharmacologic activity. Active agents that can be included in the composition include, for example, corticosteroids and beta-2 agonists. Corticosteroids include, but are not limited to, beclomethasone, budesonide, ciclesonide, flunisolide, fluticasone, mometasone, rofleponide, triamcinalone, terbutaline. Beta-2 agonists including, but are not limited to albuterol, bitolterol, fenoterol, formoterol, isoetharine, isoproterenol, metaproterenol, salmeterol, xinofoate and pirbuterol. In another embodiment, the composition further comprises a beta-2 agonist. In a further embodiment, the beta-2 agonist is selected from the group consisting of formoterol and salmeterol.

The inventive pharmaceutical composition comprising a dry powder can be administered by inhalation using an inhalation device. Dry powder formulations as described herein may be delivered using any suitable dry powder inhaler (DPI), i.e., an inhaler device that utilizes the patient's inhaled breath as a vehicle to transport the dry powder drug to the lungs. Examples of suitable inhalers include those of United States Pat. Publication No. 2003/0150453, and PCT publication WO 02/083220 which are hereby incorporated by reference. Other examples include dry powder inhalation devices as described in U.S. Pat. Nos. 5,458,135; 5,740,794 and 5,785,049, all herein incorporated by reference. When administered using a device of this type, the powdered composition is contained in a receptacle having a puncturable lid or other access surface, preferably a blister package or cartridge, where the receptacle may contain a single dosage unit or multiple dosage units. Convenient methods for filling large numbers of cavities (i.e., unit dose packages) with metered doses of dry powder medicament are described, e.g., in PCT Publication No. WO 97/41031, incorporated herein by reference.

Other dry powder dispersion devices for pulmonary administration of dry powders include those described, for example, in European Patent No. EP 129985, European Patent No. EP 472598, European Patent No. EP 467172, U.S. Pat. No. 5,522,385, all of which are incorporated herein by reference. Also suitable for delivering dry powder formulations described herein are inhalation devices such as the Astra-Draco “TURBUHALER”. This type of device is described in detail in U.S. Pat. Nos. 4,668,218; 4,667,668; 4,805,811, all of which are incorporated herein by reference. Other suitable devices include dry powder inhalers such as ROTAHALER® (Glaxo), DISCUS® (Glaxo), SPIROS™ inhaler (Dura Pharmaceuticals), and the SPINHALER® (Fisons). Also suitable are devices which employ the use of a piston to provide air for either entraining powdered composition, lifting medicament from a carrier screen by passing air through the screen, or mixing air with powder medicament in a mixing chamber with subsequent introduction of the powder to the patient through the mouthpiece of the device, such as described in U.S. Pat. No. 5,388,572, incorporated herein by reference.

Formulations described herein may also be delivered using a pressurized, metered dose inhaler (MDI), e.g., the VENTOLIN® metered dose inhaler, containing a solution or suspension of drug in a pharmaceutically inert liquid propellant, e.g., a chlorofluorocarbon or fluorocarbon, as described in U.S. Pat. No. 5,320,094 and in U.S. Pat. No. 5,672,581, both incorporated herein by reference.

Alternatively, the formulations described herein may be dissolved or suspended in a solvent, e.g., water or saline, and administered by nebulization. Nebulizers for delivering an aerosolized solution include the AERx™ (Aradigm), the ULTRAVENT® (Mallinkrodt), the PARI LC PLUS™ or the PARI LC STAR™ (Pari GmbH, Germany), the DeVilbiss Pulmo-Aide, and the Acorn II (Marquest Medical Products).

In addition to administration by inhalation, the inventive complex and pharmaceutical compositions may be administered orally. Forms suitable for oral administration include, for example, tablets, lozenges, hard or soft capsules, aqueous or oily suspensions, emulsions, dispersible powders or granules, syrups or elixirs. The inventive complex or pharmaceutical composition can also be administered by controlled release means and/or delivery devices such as those described in U.S. Pat. Nos. 3,845,770; 3,916,899; 3,536,809; 3,598,123 and 4,008,719; the disclosures of which are hereby incorporated by reference.

The invention is illustrated by the following examples which are not meant to be limiting in any way.

### EXEMPLIFICATION

### Example 1

### Preparation of Trospium Saccharin Complex Crystals

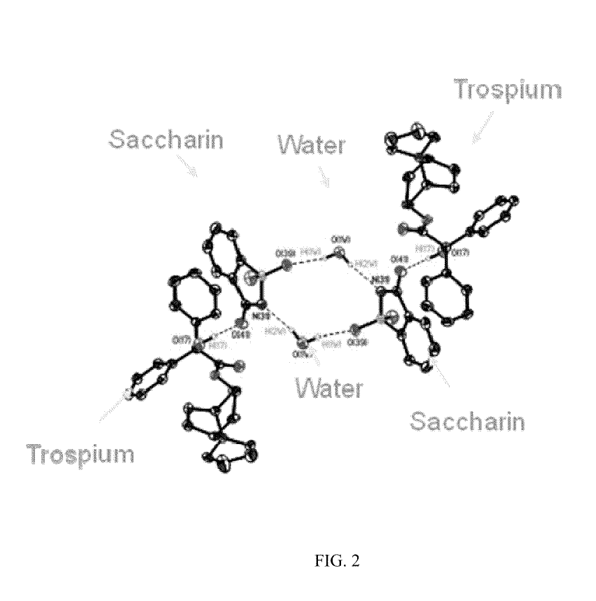

A solution containing trospium chloride and water was combined with a solution containing sodium saccharin in water. The solutions were combined at a 1:1 molecular ratio of trospium chloride:sodium saccharin (2:1 mass ratio). Upon mixing at room temperature, precipitate was visible in less than a minute. Over several minutes, the amount of solids in the suspension increased. The solids were confirmed to be crystals by microscopy. Trospium saccharin complex is soluble in water at room temperature at less than about 0.4 mg/ml. A drawing of the crystal structure for the trospium saccharin precipitate is shown in FIG. 2.

### Example 2

### Preparation of Trospium Saccharin Complex Inhalable Powders

Trospium saccharin complex inhalable powder (10% by weight trospium) was prepared by adding 1.0 g of trospium chloride to the ethanol feed of a spray dryer and adding 0.5 g of sodium saccharin to the aqueous feed of the spray dryer. 8.5 g of leucine was also dissolved in the aqueous feed. The two feeds were preheated online to 55° C. and mixed, 30% ethanol and 70% aqueous feed by volume continuously during spray drying just upstream of the spray drying head. After mixing the solvents, the total solids concentration was 10 g/L. After spray drying, the leucine-based inhation powder was harvested from the spray drier. The trospium saccharinate crystals formed in situ in the powder during spray drying. It is believed that the trospium saccharinate crystals were embedded in leucine particles. The solubility of trospium saccharin complex was greater in the mixture of ethanol and water than in water alone. Therefore, at 30% ethanol with inline heating at 55° C., the solubility of trospium saccharin complex was greater than 4 mg/ml.

Trospium saccharin complex inhalable powders (2% and 4% by weight trospium) were prepared by adding 0.2 g and 0.4 g of trospium chloride respectively, to the ethanol feed of a spray dryer and adding 0.1 g and 0.2 g of sodium saccharin respectively, to the aqueous fed of the spray dryer. 9.7 g and 9.4 g of leucine respectively, were also dissolved in the aqueous feed. The two feeds were mixed at room temperature (25° C.), 20% ethanol and 80% aqueous feed by volume continuously during spray drying just upstream of the spray drying head. After mixing the solvents, the total solids concentration was 13 g/L. After spray drying, the leucine-based inhalation powder was harvested from the spray-dryer. The trospium saccharinate crystals formed in situ in the powder during spray drying.

Trospium saccharin complex inhalable powder was prepared by adding 0.5 g of saccharin (acid) to the ethanol feed while 1.0 g of trospium chloride and 8.5 g of leucine was added to the aqueous feed. The same spray drying parameters above apply.

### Example 3

### Physical Experimental Methodology for XRPD and DSC Analyses

XRPD measurements were performed using Bruker AXS D8 Focus X-ray powder diffractometer in the θ/2θ mode. The scanning parameters were as follows: Samples are scanned from 2.5° to 40° 2θ range at the 0.02°/step with 1 second interval. The accuracy of peak positions is defined as ±0.2.degrees 2θ due to experimental differences, such as instrumentations, sample preparations, and the like. XRPD was used to characterize formulations comprising trospium and saccharin. The formulations tested were trospium saccharin precipitate; 30% (w/w) trospium/15% (w/w) sodium saccharin/55% (w/w) leucine; 10% (w/w) trospium/5% (w/w) sodium saccharin/85% (w/w) leucine; 10% (w/w) trospium/5% (w/w) sodium saccharin/10% (w/w) sodium citrate/75% (w/w) leucine and 10% (w/w) trospium/5% (w/w) acid saccharin/85% (w/w) leucine formulated powders.

The XRPD data for these formulations is shown in FIG. 1A. XRPD was also used to characterize saccharin, trospium saccharinate, sodium saccharin and trospium chloride. The XRPD data for these compounds is shown in FIG. 1B. The XRPD data for the trospium saccharin complex is shown in FIG. 1C.

Differential scanning calorimetry (DSC) measurements were performed on a TA instruments DSC Q1000 with a sample having a weight of about 2 mg (for trospium saccharinate precipitate) and 5 mg (for formulation containing saccharinate salt). The heating rate was 10° C./minute with a nitrogen stream flow rate of about 50 ml/min over a scan range of from about 20° C. to 200° C.

DSC analysis was utilized to characterize trospium saccharin complex, and formulations comprising 30, 10 and 4% trospium chloride. The DSC profile for these compounds and formulations is shown in FIGS. 3A and 3B.

### Example 4

### Moisture Challenge of Trospium Containing Formulations in Relative Humidity (RH) Equilibration Study

The moisture challenge of trospium containing formulations shown in Table 1 below was determined in an RH equilibration study.

| | | | Solvent | | |
|---|---|---|---|---|---|
| Formulation | Formulation | | Ratio | Organic | Conc. |
| type | Ratio (wt %) | Formulation | (Org/Aq) | Solvent | (g/L) |
| DPPC | 85/10/5 | Leucine/Trospium/DPPC | 70/30 | Ethanol | 5 |
| Low Ethanol | 90/10 | Leucine/Trospium | 30/70 | Ethanol | 10 |
| | | Leucine/Sodium | | | |
| Citrate | 80/10/10 | Citrate/Trospium | 30/70 | Ethanol | 10 |
| Acetone | 90/10 | Leucine/Trospium | 20/80 | Acetone | 10 |
| Saccharin | 85/10/5 | Leucine/Trospium/Sodium | 30/70 | Ethanol | 10 |
| | | Saccharin | | | |

Briefly, 200 mg of each sample was weighed and placed into open glass scintillation vials and equilibrated overnight at 20, 40 and 60% RH in a glove box at 25° C. The following day, the vials were capped and sealed into aluminum pouches in the glove boxes under the RH conditions. A portion of the sample in the vials was then placed at −20° C., in a 50° C. incubator or a 60° C. incubator. After one week at either 50° C. or 60° C., sample were removed from the incubator and then equilibrated to room temperature and placed at −20° C. Samples were then removed from the −20° C., equilibrated for at least one hour and the geometric particle size of the emitted powder was measured for each of the samples. The percent increase in volume median geometric diameter (VMGD) of the emitted powder was plotted relative to the initial VMGD of the sample that had been equilibrated at 20% RH (20% RH overnight equilibration sample VMGD was subtracted from the other treated sample's VMGD and the difference was then divided by the VMGD of the 20% RH overnight equilibration sample then multiplied by 100 to obtain percent increase). The data from these studies is shown in FIGS. 4A, 4B and 4C. The saccharin formulation showed no significant change in VMGD at the RHs and temperatures tested.

### Example 5

### Pharmacokinetic (PK) Profile of Trospium Containing Powder Formulation Administered to Rats Via Insufflations

Male Sprague-Dawley rats (weight 400±50 grams) were administered various powder formulations of trospium by insufflation. The rats were divided into four groups of four rats as follows:
Group A—Insufflated intratracheally with Leucine/Trospium/DPPC containing approximately 200 μg Trospium
Group B—Insufflated intratracheally with Leucine/Trospium/NaSaccharin containing approximately 200 μg Trospium
Group C—Insufflated intratracheally with Leucine/Citrate/NaSaccharin/Trospium containing approximately 200 μg Trospium
Group D—Insufflated intratracheally with Leucine/Trospium containing approximately 200 μg Trospium
   The formulations tested are described in Table 2.

TABLE 2

| Formulation | | EtOH/Aqueous | Solids |
|---|---|---|---|
| Ratio | Formulation | Ratio | (g/L) |
| 85/10/5 | Leucine/Trospium/DPPC | 70/30 | 5 |
| 85/10/5 | Leucine/Trospium/ | 30/70 | 10 |
| | NaSaccharin | | |
| 75/10/5/10 | Leucine/Citrate/ | 30/70 | 10 |
| | NaSaccharin/Trospium | | |
| 90/10 | Leucine/Trospium | 30/70 | 10 |

Rats were anesthetized using inhaled Isofluorane. Drug powder was then intratracheally insufflated into each rat. All animals were allowed food and water ad libitum between blood collection time points. Blood samples were collected by a lateral tail vein after anesthesia. A syringe without an anticoagulant was used for blood collection and the whole blood was transferred to tubes containing K2 EDTA (MIRCOTAINER®; MFG# BD365974). The blood samples were processed (the tubes are inverted 15-20 times and centrifuged for 2 minutes at >14,000 g's to separate plasma). The plasma samples prepared in this manner were transferred to labeled plain tubes (MICROTAINER®; MFG# BD5962) and stored frozen at <−70° C. 250 ul of whole blood was obtained for each time point. Sample collection times were pre-insufflation, 2.5, 5.0, 7.5, 10, 15, 30, 60 and 120 minutes after insufflation.

Plasma samples were analyzed for trospium using an Ionspray LC/MS/MS (Liquid Chromatography coupled with Mass Spectrometery). Briefly, 100 ul of standard (trospium standard solutions in rat plasma), control or test samples were added to the wells of a 96-well plate. The trospium standard solutions were prepared by adding 100 ul of 1 ug/ml trospium chloride (in methanol) with rat plasma to yield a nominal concentration of 4000 pg/ml. Standard solutions in rat plasma were prepared as follows:

| Standard, pg/ml | Volume (ul) of 4000 pg/ml | Volume (ul) of |
|---|---|---|
| in rat plasma | standard in rat plasma | rat plasma |
| 4000 | 100 | 0 |
| 2000 | 50 | 50 |
| 1000 | 25 | 75 |
| 500 | 12.5 | 87.5 |
| 250 | 12.5 | 187.5 |
| 100 | 5 | 195 |
| 50 | 2.5 | 197.5 |
| 0 | 0 | 100 |
| blank | 0 | 100 |

Controls were prepared by adding 1 ug/ml trospium chloride in methanol to rat plasma to yield concentrations of 2500 pg/ml, 800 pg/ml and 200 pg/ml of internal standard solution (2.5 ng/ml clidinium bromide in water) were added to all wells except those containing blank plasma samples. 20 ul of 50 mM ammonium acetate (pH 9.0) solution and 350 ul acetonitrile were added to all wells and the plate was shaken on an orbital shaker for 1-2 minutes. The plate was centrifuged at 2000×g for 5-7 minutes. The supernatant was removed added onto an Agilent 96-well plate and concentrated to dryness using a Speed Vacuum. The dry sample were then reconstituted with 100 ul of 50:50 methanol:water and analyzed by LC/MS/MS.

The HPLC operating conditions were as follows:

| Mobile phase | 30% 5 mM ammonium acetate buffer: | |
|---|---|---|
| | 70% methanol | |
| Flow rate | 250 | ul/min |
| Injection volume | 5 | ul |
| Run time | 3 | min |

The MS/MS operating conditions were as follows:

| | |
|---|---|
| Trospium transition | m/z 392−>164 |
| Clidinium transition | m/z 352−>142 |
| Scan mode | Multiple reaction monitoring (MRM) |
| Monitoring | Positive ion mode |

Quantitation was performed using a weighted (1/×) linear regression analysis generated from standard samples.

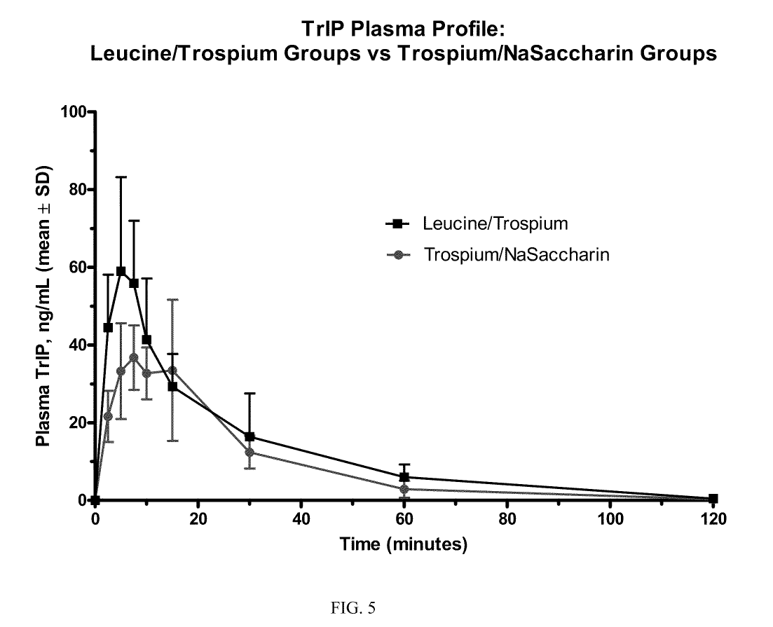

The results of the study are shown in FIG. 5 and relevant PK parameters are summarized below in Table 3.

TABLE 3

| | Trospium groups | | |
|---|---|---|---|
| | (mean ± standard | Trospium saccharin | |
| Measure | error) | precipitate groups | P value |
| Cmax (ng/ml) | 71.25 ± 6.26 | 43.50 ± 5.32 | <0.005 |
| Tmax (min) | 6.31 ± 0.83 | 9.38 ± 1.40 | <0.08 |
| AUC (ng min/ml) | 1493 ± 112.6 | 1102 ± 76.9 | <0.01 |

### Example 6

### Agglomeration of Trospium Powder Formulations

The trospium powder formulations described in Table 4 were prepared as described in Example 2 in order to compare particle agglomeration. The results of this study are summarized below in Table 4.

TABLE 4

| | Online VMGD during | Recovered bulk powder | |
|---|---|---|---|
| Formulation | spray drying (um) | mean VMGD (um % RSD) | |
| 2% (w/w) trospium | 6.4 | 9.6 | (3.9) |
| (TrCl/Sodium | 6.2 | 9.3 | (3.6) |
| saccharin/Leucine) | | | |
| 2% (w/w) trospium | n/a | 15.8 | (2.2) |
| (TrCl/Leucine) | 7.2 | 15.0 | (2.2) |
| 4% (w/w) trospium | 6.0 | 8.5 | (3.4) |
| (TrCl/sodium | 5.9 | 8.7 | (4.4) |
| saccharin/leucine) | | | |
| 4% (w/w) trospium | 6.3 | 13.7 | (2.6) |
| (TrCl/Leucine) | 6.8 | 14.8 | (3.0) |

As shown in Table 4, powders comprising 2 and 4% (w/w) trospium and sodium saccharin show reduced particle agglomeration compared with powders in the absence of sodium saccharin.

### Example 7

### Evaluation of the Efficacy and Pharmacokinetics of Trospium Inhalation Powder (TrIP) Administered to Subjects with Chronic Obstructive Pulmonary Disease (COPD)

The objectives of this study were to assess the efficacy and pharmacokinetics (PK) of inhaled administration of TrIP to subjects with moderate to severe COPD. This was a single-center, randomized, double-blind, cross-over, placebo-controlled study. The study included 24 male and female subjects with COPD, aged 40 to 80.

Following a screening visit, each subject was randomized to a dosing sequence. Study subjects received a total of 5 doses, each separated by a 3- to 14-day washout period. Doses A, B, C, and D were administered in a double blind fashion, in sequences generated by a 4-period Latin square design. Each subject had 6 visits over a period of approximately 2 to 10 weeks.

Two different trospium chloride (TrCl) formulations were used in this study, TrIP-2D and TrIP-2SS. Both formulations were supplied as a dry powder and packaged into size 2 capsules (“active” capsule) for inhalations using the C2S inhaler (one capsule/inhaler). The TrIP-2D formulation is composed of 2% TrCl (100 μg) formulated with leucine and 1,2-dipalmitoyl-sn-glycero-3-phosphocholine (DPPC). The TrIP-2SS formulation is composed of 2% TrCl (100 μg) formulated with leucine and sodium saccharin. The administered doses were as follows:

Dose A=placebo, 4 empty size-2 capsules

Dose B=TrIP-2D (100 μg TrCl), 1 active capsule and 3 empty placebo capsules

Dose C=TrIP-2SS (100 μg TrCl), 1 active capsule and 3 empty placebo capsules

Dose D=TrIP-2D (400 μg TrCl), 4 active capsules

Blood collection for PK analysis occurred on visits 2, 5, and 6 for all subjects.

Blood samples were collected at predose (0) and at 2, 5, 15, 30 minutes and 1, 2, 3, 4, 6, 8, 12, and 24 hours postdose. Plasma samples were analyzed by CEDRA Corporation (Austin, Tex.) using validated LC-MS-MS procedure. Pharmacokinetics was evaluated on the basis of:
Maximum plasma concentration (Cₘₐₓ) of trospium
Time to Cₘₐₓ (Tₘₐₓ)
Area under the plasma concentration time curve such as AUC_{0-t last} and AUC_{0-∞} (or as appropriate)

A summary of the pharmacokinetic parameters of Dose B, C and D is provided in Table 5.

TABLE 5

| | TrIP-2D | | TrIP-2D | | TrIP-2SS | |
|---|---|---|---|---|---|---|
| | 100 ug (12) | | 400 μg (12) | | 100 μg (12) | |
| Treatment | DOSE B | | DOSE C | | DOSE D | |
| (N) | Mean | SD | Mean | SD | Mean | SD |
| Cₘₐₓ | 279 | 202 | 1020 | 779 | 283 | 159 |
| (pg/mL) | | | | | | |
| Tₘₐₓ (min)^{a} | 5.0 | 2.0-5.0 | 5.0 | 2.0-5.0 | 5.0 | 2.0-30.0 |
| AUCₗₐₛₜ | 214 | 149 | 991 | 639 | 262 | 144 |
| (pg/mL * hr) | | | | | | |

^{a}Tₘₐₓ expressed as median and range

Results indicate a similar Cₘₐₓ and AUCₗₐₛₜ across all 100 μg doses regardless of treatment and trospium exposure increasing with increasing dose.

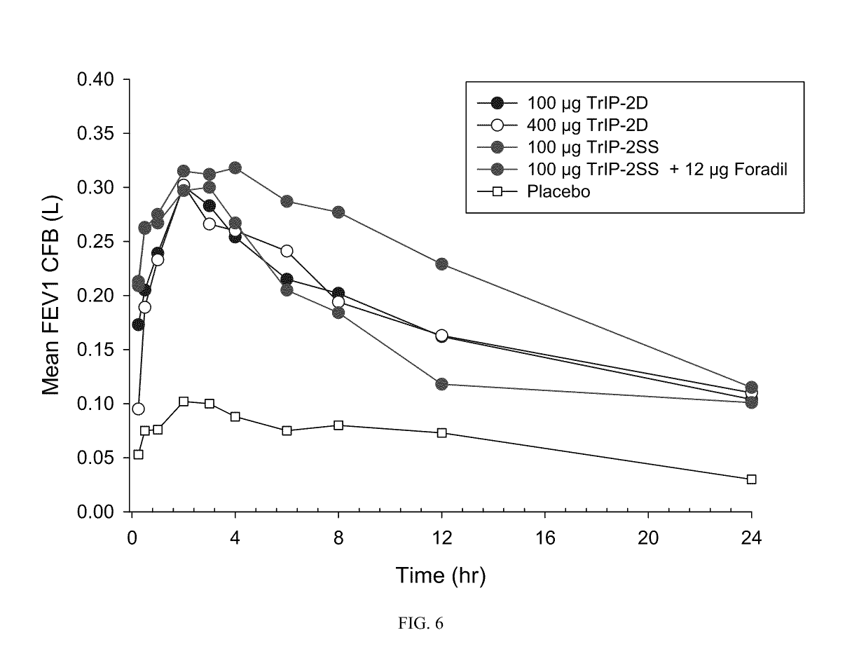

Spirometry measurements included forced expiratory volume in one second (FEV₁), forced vital capacity (FVC), FEV₁/FVC, and % predicted FEV₁. These measurements were taken both at screening and at all dosing visits at predose (0), and at 15 and 30 minutes, 1, 2, 3, 4, 6, 8, 12, and 24 hours postdose. Spirometry throughout the study was performed following American Thoracic Society spirometry guidelines. Efficacy was evaluated on the basis of:
Spirometry measurements, including, but not limited to, peak FEV₁, average FEV₁ over 24 hours, FEV₁ at postdose time points, FEV₁/FVC, % predicted FEV₁, time to onset of response (with response defined as FEV₁≧12% or 200 mL above baseline), and FEV₁ change from baseline (CFB) at postdose time points (FIG. 6).

All treatment groups resulted in significant increase in mean FEV₁ change from baseline in comparison to placebo, which was maintained out to 24 hours postdose. The 100 μg TrIP dose was maximally effective with no apparent difference between the two formulations.

### Example 8

### Trospium Saccharinate Stability Study

Compositions containing 2% trospium saccharinate (2% TrCl-SS) were subjected to stress conditions of 40±2° C./75% RH±5% for 0, 1, 3 and 6 months. The powder appearance, related impurities, moisture content, emitted dose and aerodynamic particle size distribution (aPSD) were measured at 0, 1, 3, 6 months.

Physical powder appearance was determined by visual inspection. Samples were removed from stress conditions, allowed to equilibrate to room temperature and inspected for sample color, clarity and any visible signs of foreign particulate matter.

Impurities were assessed using an HPLC method. TrCl-SS samples were prepared at a concentration of 70 μg trospium/mL of solution. The injection volume was 150 μl. All samples were prepared in duplicate.

Water content was determined using a Brinkmann (Metrohm) 756 Karl Fischer Coulometer with a 774 oven sample processor.

The mean emitted dose is determined using the emitted dose apparatus. Reversed-phase HPLC was used to quantify the 2% TrCl-SS sample content in the emitted portion. A flow meter and pump were connected to the emitted dose apparatus and the flow was adjusted to 28.3 L/min. The inhaler was loaded with a capsule containing the 2% TrCl-SS sample composition, the capsule was punctured, and the inhaler was attached to the emitted dose apparatus using a mouthpiece adapter. The pump was activated for an appropriate duration to achieve a total volume of 2 L, dispersing the 2% TrCl-SS powder onto a filter disk housed in the emitted dose apparatus. The 2% TrCl-SS powder was recovered from the filter and the mass of TrCl-SS recovered was determined by reversed-phase HPLC.

Aerodynamic Particle Size Distribution (aPSD) of the Emitted Dose for 2% TrCl-SS compositions was determined using an Andersen Cascade Impactor (ACI). Reversed-phase HPLC was used to quantify the TrCl-SS content. A flow meter and pump were connected to the ACI and the flow was adjusted to 28.3 L/min. The inhaler was loaded with a capsule containing the sample composition, the capsule was punctured, and the inhaler was attached to the induction port using a mouthpiece adapter. The pump was activated for an appropriate duration to achieve a total volume of 2 L, dispersing the 2% TrCl-SS powder. A solution containing 0.01 N HCl was used to recover 2% TrCl-SS powders from the relevant components of the ACI, and the 2% TrCl-SS content was determined by reversed-phase HPLC.

Table 6 is a summary of 40° C./75% RH accelerated stability data for 2% TrCl-SS compositions.

TABLE 6

| Test | Method | Specifications | Initial | 1 month | 3 month | 6 month |
|---|---|---|---|---|---|---|
| Appearance | 110-00722 | Clear capsule containing white to | Conforms | Conforms | Conforms | Conforms |
| | | off-white powder, no visible foreign | | | | |
| | | particulate matter | | | | |
| Assay - Assay (% CS) | 110-02511 | 90.0 to 110.0% | 105.5% | 97.8% | 99.5% | 99.8% |
| Assay - Assay (μg | 110-02511 | 90 to 110 μg | 105 μg | 98 μg | 100 μg | 100 μg |
| TrCl) | | | | | | |
| Related Impurities - | 110-02444 | ≧98.0% | 100.0% | 99.9% | 99.9% | 99.9% |
| TrCl Purity | | | | | | |
| Related Impurities - | 110-02444 | ≦1.0% | 0.0% | 0.0% | 0.0% | 0.0% |
| Benzilic Acid | | | | | | |
| Related Impurities - | 110-02444 | ≦1.0% | 0.0% | 0.1% | 0.1% | 0.1% |
| Unidentified | | | | | | |
| Impurities, Total | | | | | | |
| Related Impurities - | 110-02444 | ≦0.5% | 0.0% | 0.1% | 0.0%, 0.0%, 0.0%, | 0.0%, 0.0%, 0.0%, |
| Unidentified | | | | | 0.0% | 0.0% |
| impurities, Individual | | | | | | |
| Related Impurities - | 110-02444 | ≦2.0% | 0.0% | 0.1% | 0.1% | 0.1% |
| Total Impurities | | | | | | |
| Water Content | 110-02772 | ≦3.0% | 0.1% | 0.1% | 0.1% | 0.1% |
| Emitted Dose - Emitted | 110-02468 | Report Result Alert if outside | 91% CS | 88% CS | 91% CS | 87% CS |
| Dose Mean (% CS) | | 70-110% of capsule strength (CS) | | | | |
| | | Action if outside 60-120% of CS | | | | |
| Emitted Dose - Emitted | 110-02468 | Report Result Alert if outside 70-110 μg | 91 μg | 88 μg | 91 μg | 87 μg |
| Dose Mean(μg) | | Action if outside 60-120 μg | | | | |
| aPSD - aPSD Mean - | 110-02467 | ≧35% | 70% | 64% | 65% | 63% |
| FPF < 5.8 μm (%) | | | | | | |

Results indicate that the formulation is both chemically and physically stable over 6 months at the stress conditions.

While this invention has been particularly shown and described with references to preferred embodiments thereof, it will be understood by those skilled in the art that various changes in form and details may be made therein without departing from the scope of the invention encompassed by the appended claims.

What is claimed:

1. A complex of trospium and saccharin.

2. The complex of claim 1, wherein the complex is a salt.

3. The complex of claim 1 which is in a crystalline form.

4. The complex of claim 2 which is a monohydrate.

5. The complex of claim 3 which is a monohydrate.

6. The complex of claim 1 which is a particulate.

7. The complex of claim 1 characterized by at least two major XRPD peaks at 9.7 degrees 2θ and 15.4 degrees 2θ±0.2 degrees 2θ.

8. The complex of claim 1 characterized by an endothermic transition between about 160° C. and about 185° C. in the DSC profile.

9. A pharmaceutical composition comprising a complex of trospium and saccharin in a therapeutically effective amount and a pharmaceutically acceptable carrier or excipient.

10. The composition of claim 9, wherein the composition is a particulate.

11. The composition of claim 9, wherein the composition is a dry powder.

12. The composition of claim 9, wherein the complex is a micronized form.

13. The composition of claim 9, wherein the complex is a spray-dried form.

14. The pharmaceutical composition of claim 9 adapted for use with a dry powder inhaler.

15. The composition of claim 10 having a density of less than about 0.4 g/cm³.

16. The composition of claim 10, wherein the composition has a mean mass aerodynamic diameter of about 1 to about 5.8 microns.

17. The composition of claim 10 further comprising one or more additional agents selected from the group consisting of a bulking agent, a flavoring agent and a phopholipid.

18. The composition of claim 10, wherein the additional agent is an amino acid.

19. The composition of claim 18, wherein the amino acid is a hydrophobic amino acid.

20. The composition of claim 19, wherein the amino acid is leucine.

21. The composition of claim 9 comprising between about 1 to about 10% by weight of the complex of trospium and saccharin.

22. The composition of claim 18 comprising between about 1 to about 10% trospium saccharin complex.

23. The composition of claim 22 comprising between about 85 to about 99% leucine.

24. The composition of claim 9, wherein the composition achieves steady state blood levels at a once daily dose of less than 100 μg.

25. A method of preparing a complex of trospium and saccharin comprising combining an aqueous solution of trospium chloride with an aqueous solution of saccharin or a salt thereof and recovering the trospium saccharin complex.

26. The method of claim 25, wherein the saccharin and trospium chloride are mixed at approximately a 1:1 molecular ratio.

27. A method of preparing a complex of trospium and saccharin comprising spray drying wherein an organic solvent and an aqueous solvent are used to form a feed wherein:
a. Trospium chloride is dissolved in the aqueous phase and saccharin or salt thereof is dissolved in the organic phase; or
b. Trospium chloride is dissolved in the organic phase and saccharin or salt thereof is dissolved in the aqueous phase.

28. A method of treating a smooth muscle hyperactivity disorder in a patient in need thereof comprising administering to said patient a therapeutically effective amount of a complex of saccharin and trospium.

29. The method of claim 28, wherein the smooth muscle hyperactivity disorder is overactive bladder.

30. The method of claim 28, wherein the complex is in the form of a dry powder.

31. The method of claim 28, wherein the complex is administered by inhalation.

32. A pharmaceutical composition comprising saccharin or a salt thereof and trospium chloride in a therapeutically effective amount and a pharmaceutically acceptable carrier or excipient.

33. The method of claim 32, wherein the composition is a powder.

34. The composition of claim 32, further comprising leucine.

35. The composition of claim 32, comprising about 0.5 to about 15% by weight trospium chloride, from about 0.1 to about 10% by weight sodium saccharin and from about 75 to about 99.4% by weight leucine.

36. The composition of claim 35, comprising about 1% by weight trospium chloride, about 0.5% by weight sodium saccharin and about 98.5% by weight leucine.

37. The composition of claim 32, comprising from about 5 to about 15% by weight trospium chloride, from about 2 to about 10% by weight sodium saccharin and from about 75% to about 93% by weight leucine.

38. The composition of claim 37, comprising about 10% by weight trospium chloride, about 5% by weight sodium saccharin and about 85% by weight leucine.

39. A method of treating a patient suffering from a condition selected from the group consisting of condition selected from the group consisting of acute lung injury (ALI), acute respiratory distress syndrome (ARDS), chronic obstructive pulmonary disease (COPD), chronic obstructive airway disease (COAD), chronic obstructive lung disease (COLD), chronic bronchitis, emphysema, bronchiectasis, pneumoconiosis and interstitial cystitis comprising administering to said patient a therapeutically effective amount of a complex of trospium and saccharin.

40. The method of claim 39, wherein the condition is COPD.

41. The method of claim 39, wherein the complex is in the form of a dry powder.

42. The method of claim 39, wherein the complex is administered by inhalation.

43. A method of treating a patient suffering from a condition selected from the group consisting of condition selected from the group consisting of acute lung injury (ALI), acute respiratory distress syndrome (ARDS), chronic obstructive pulmonary disease (COPD), chronic obstructive airway disease (COAD), chronic obstructive lung disease (COLD), chronic bronchitis, emphysema, bronchiectasis, pneumoconiosis and interstitial cystitis comprising administering to said patient the pharmaceutical composition of claim 32.
